(19) United States
(12) Patent Application Publication
(10) Pub. No.: US 2015/0147555 A1
(43) Pub. Date: May 28, 2015

(54) MULTILAYER MUCOADHERING PATCH WITH A NONADHERING LAYER COMPRISING CASEIN

(71) Applicant: OraHealth Corporation, Bellevue, WA (US)

(72) Inventor: Jeffrey T. Haley, Mercer Island, WA (US)

(21) Appl. No.: 14/550,969

(22) Filed: Nov. 22, 2014

Related U.S. Application Data
(60) Provisional application No. 61/908,047, filed on Nov. 23, 2013.

(51) Int. Cl. A61L 15/32; A61K 9/70

(52) U.S. Cl. CPC A61L 15/32; A61K 9/70
USPC 428/220; 428/343

(57) ABSTRACT

A multilayer, mucoadhering patch, comprising a first, exposed layer of adhesive material including a polymer that is capable of adhering to a wet mucous surface in a human or other animal; and a second, exposed layer of nonadhesive material comprising casein.

### CROSS-REFERENCE TO RELATED APPLICATIONS

This application claims the benefit of U.S. Application No. 61/908,047, filed 23 Nov. 2013, which is incorporated in its entirety.

### TECHNICAL FIELD

The present disclosure relates to a mucoadhering patch capable of being to be adhered to a wet mucous surface of a mucous membrane in humans or other animals. More particularly, it relates to a multilayer mucoadhering patch that has improved mouth feel and slipperiness on the nonadhesive side.

### SUMMARY OF THE INVENTION

The present disclosure is directed to a multilayer, mucoadhering patch comprising (a) a first, exposed layer of adhesive material including a polymer that is capable of adhering to a wet mucous surface in a human or other animal; and (b) a second, exposed layer of nonadhesive material comprising casein.

In some embodiments, the concentration of casein is at least 40% by weight of the nonadhesive layer. In other embodiments, the concentration of casein is at least 80% by weight of the nonadhesive layer. In certain embodiments, casein is selected from the group consisting of acid casein, rennet casein, fresh curd casein, micellar casein, calcium caseinate and sodium caseinate.

The patch may comprise two layers. In certain embodiments, at least one of the layers is a pressed powder. In other embodiments, the first layer is a pressed powder and the second layer is painted onto one side of the first layer. In yet other embodiments, the first layer and the second layer are bonded together. The patches may be cut from sheets of the first and second layers that are bonded together. In other embodiments, the first and second layers are cast or deposited as a liquid or viscous material and dried or otherwise cured together. The casein may be dusted as a powder, then misted with water, and then dried.

The patches may be any shape and generally have a flattish shape. Orthogonal dimensions of the patch are both 5-18 mm. Kits may be provided, in which kits comprise patches and instructions for use.

### DETAILED DESCRIPTION

The present disclosure provides a multilayered patch that has at least two layers, a first layer capable of adhering to a mucosal surface, and a second layer that comprises casein and is nonadherent. In certain embodiments, the patch further comprises an active ingredient. Typically, the patch is designed to release a medicament or other active ingredient in the localized area of the patch over a relatively long period of time.

As used herein, a “patch” is an adhesive device that can adhere to a mucosal or otherwise wet surface in a human or other animal. In certain embodiments, the patch can adhere to gums or teeth, structures attached to teeth (e.g., orthodontic braces), or vaginal, vulval, labial, rectal, anal, or oral mucosal tissue.

A multilayered patch comprises at least two layers. A first layer is capable of adhering to a mucosal surface in a human or other animal. A second layer is nonadherent, comprises casein, and is exposed on the surface of the patch. Any additional layers are positioned in between the first and second layers, and may or may not be exposed on the edges of the patch. In embodiments wherein the patch comprises an active ingredient, it may be contained in any or all of the layers or any subset of the layers, although generally, it will be in either the first or second layer.

A patch may be any shape, including round (when viewed from above or below), rectangular, ovoid, other polygonal shape, or irregularly shaped. The shape is chosen at least in part by the manufacturing process. Typically, the length of orthogonal dimensions is in the range of about 5 to about 18 mm, and the thickness from about 1 mm to about 10 mm. In some embodiments, the patch may have a dimension as large as 30 mm. The surfaces of a patch can be flat, essentially flat, convex, concave, dimpled, domed, or irregular. In certain embodiments, the two surfaces have different shapes, such as one side being convex and the other side being flat or having a dimple (concave). A patch with a dimple on the adherent side is exemplified in U.S. applications Ser. Nos. 12/287,647 and 12/325,194, which are incorporated for disclosure of a dimpled patch and manufacturing methods. The layers can be different volumes and weights or similar volumes and weights. Likewise, the thickness of the layers can be different or similar. The size of the nonadherent layer can be larger than the adherent layer, such that the surface of the adherent layer is e.g. “ringed” by the nonadherent layer, as long as the patch is able to adhere to a mucosal surface.

The disclosed patches comprise casein in the second layer, which is nonadherent. Casein exhibits several desirable characteristics, including imparting a slippery feel to a patch, long dissolution (erosion) time, yet fully dissolving, flexible yet strong, and a natural product. Casein is a protein molecule made by mammary glands. It is a primary food source for infant mammals and the primary solid component of milk, yogurt, and cheese. It may be purified from the milk of any mammal such as beef cows. Casein is the name for a family of phosphoproteins. There are four main types of casein, which make up approximately 80% of the total protein in bovine milk: α-s1 Casein, α-s2 Casein, β-Casein, and γ-Casein. While any of these caseins is suitable for the disclosed patches, typically, a naturally occurring mixture of the four types will be used. Casein may be extracted from milk by well-known methods or obtained from a commercial source, usually as a dried powder in forms called fresh curd, micellar, acid casein, rennet casein, calcium caseinate, and sodium caseinate. By mixing with water, forming a shape, and drying, casein can make a strong structure of any shape including a film or blocks from which shapes such as buttons may be milled. In certain embodiments, a plasticizer such as glycerin may be added to keep the dried casein soft and flexible.

In the final patch product, the concentration of casein is typically at least 40% by weight of the nonadherent layer. In some embodiments, the casein concentration is at least 50% by weight, or at least 60% by weight, or at least 70% by weight, or at least 80% by weight or at least 90% by weight of the nonadherent layer.

A multilayered patch may comprise other ingredients. In some embodiments, a patch comprises an active ingredient, such as a medicament or vaccine. Active ingredients can be any substance that treats or ameliorates a condition or disease, that prevents a condition or disease, that provides a pleasurable experience for the user (e.g., a flavor), etc. Other types of ingredients include for example, one or more adhesives (e.g., acacia gum), one or more binders (e.g., cellulose gum, acacia gum), one or more flavorings (e.g., licorice), one or more vitamins, one or more coloring agents, one or more corrigents, one or more flavors, one or more excipients (e.g., salts, buffers), one or more viscous and lubricating substances (e.g., cellulose gum), and one or more adjuvants. In the nonadherent layer, other ingredients may include one or more lubricants or one or more glidants. In some embodiments only one additional ingredient may be present, in other embodiments, multiple other ingredients may be present. Compositions of other ingredients are well-known in the art.

To facilitate the patch dissolving (eroding) slowly, a binder that dissolves slowly is optionally incorporated. Some suitable binders include carrageenan (especially kappa form), xanthan gum, xanthan gum combined with konjac gum, agar, and cellulose gums such as carboxymethylcellulose (CMC), hydroxyproplycellulose (HPC) and hydroxyproplymethylcellulose (HPMC). Other gums similar to those listed, such as locust bean gum which has properties similar to konjac gum, and guar gum are also suitable, as well as starches, such as corn starch or, particularly pregelatinized corn starch. In addition, the binder may also moderate any strong flavors by spreading out over a long period of time the release of that flavor. Consequently, sweeteners and other products to mask strong flavors are not required, although some users may prefer a small amount of sweetener and some may prefer the addition of other flavors.

The adherent layer comprises one or more natural or synthetic polymers which have adhesiveness to the wet mucous surface or a combination of said polymers and, in some embodiments, an active ingredient, such as a medicament. Adhesive molecules include acacia gum, gelatin, alginate, starch, pectin, polyvinylpyrolidone, carboxymethylcellulose, hydroxymethylcellulose, polyvinyl acid, polyacrylic acid, and carbopol. Concentrations of these adhesive molecules are well-known.

The nonadhesive layer should have no significant adhesiveness to a wet mucous surface. Furthermore, it is water soluble or water disintegrable and comprises casein. A nonadhesive layer can comprise an active ingredient. In embodiments where the nonadhesive layer comprises an active ingredient, when the preparation is adhered to a wet mucous surface, the nonadhesive layer gradually dissolves or disintegrates due to the presence of secretion, thus slowly releasing the active ingredient. The layer may comprise other ingredients that aren't adhesive to a mucosal surface or are present at a concentration too low to be adhesive. For example, other ingredients can include non-dissolving plastic such as polyethylene film, food gums (e.g., gelatin and carboxymethylcellulose), nonadhering carbohydrates (e.g. lactose, glucose, sucrose, starch, crystalline cellulose, dextrin, cyclodextrin), silicic acid anhydride, aluminum silicate, talc, calcium stearate, magnesium stearate, beeswax, polyethylene glycol, and polyphosphate.

The nonadhesive layer may consist of casein or comprise casein in the form of a mixture comprising two or more ingredients. Casein may be incorporated into the nonadherent layer by a variety of methods. The adherent and non-adherent layer or layers can be bonded together by compression, curing of one or the other or both. As used herein, “bonding” means sticking together and does not necessarily involve a “glue.” Curing can be accomplished by any of a number of techniques, including cooling of a thermo softened material, drying of a solvent sensitive material, or chemical interaction such as polymerization or cross-linking. Some of the methods for making a patch include the following.

Any form of dried powder casein may be pressed with a bilayer tablet press into a layer. Pressed powder casein will gain strength when it contacts water from saliva in the mouth or from mucous; when exposed to water, grains of casein powder bond to each other and form a flexible layer of suitable strength.

Alternatively, casein powder may be dusted onto a substrate, misted with water or other liquid sintering agent so the grains will sinter (bond) to each other, and then dried. The liquid may include a nonevaporating plasticizer such as glycerin. This forms a very strong and flexible layer which can be quite thin. The substrate may be a patch of adhesive material or it may be a sheet of adhesive material from which patches are cut after the casein layer is adhered.

As a further alternative, a layer of casein assembled as a dry powder may be misted with water or other liquid sintering agent to cause sintering and then dried to increase the strength of the casein layer. The liquid may include a plasticizer such as glycerin. The layer before misting with water may be a pressed patch or it may be a spread sheet from which patches are cut after the casein layer is dried. The drying process tends to cause curling of the layer, and it may physically be held flat while drying to prevent curling.

As another alternative, the casein powder may be mixed with enough water or other liquid sintering agent to form a thick, crumbly paste that is then squashed into a continuous layer or disc and held flat or in any other desired shape while it is dried. The water may include a plasticizer such as glycerin. The drying may be accelerated with heat or vacuum or both.

As another alternative, the casein powder may be mixed with more water or other liquid carrier to form a paint which is cast or deposited as a liquid or viscous material and dried or otherwise cured together with the first layer, or brushed or screen printed or sprayed or ink-jetted or rubber-transfer printed onto a substrate and then allowed to dry. The water may include a plasticizer such as glycerin.

Multilayer patches can be provided as kits comprising patches and instructions for use. Patches are designed to be placed on a mucosal surface of a human or other animal. The patch is used by adhering to a wet mucous surface of a mucous membrane of humans or other animals, and then the patch having a nonadhering layer comprising casein and an active ingredient dissolve or disintegrate over a suitable period of time. For many applications, a desired time for dissolution is at least 30 min and may be as long as 8 hours. The patch may be applied during the daytime or nighttime. Depending on the active ingredient, multiple applications may be appropriate. Either or both sides of the patch may release an active ingredient, such as a medicament or a vaccine.

The following examples are illustrative of the present disclosure.

### EXAMPLES

### Example 1

### Patches Made with a Bilayer Tablet Press

In this example, polymer powder, which is used as an adhesive ingredient, a desired active ingredient, such as a medicament, and optionally other ingredients are used to make a patch with a bilayer tablet press.

Polymer powder and a medicament, along with other ingredients added as the case may require, are thoroughly mixed to obtain an intimate powdery mixture. An intimate powdery mixture means a state of mixture in which the respective ingredients contained in the preparation are mixed most uniformly and free from maldistribution. This powder may also be made into slag and ground into granules. The adhesive may be used in pure form which avoids a need for mixing or grinding. An suitable amount for the press of the mixture or granules is processed in a bilayer tablet press.

A mixture of ingredients that forms a nonadhesive layer is simultaneously processed in the bilayer press to make a two layer tablet (patch). At the time of pressing, if the second pressing is effected with higher pressure than the first pressing, separation of the nonadhesive layer from the adhesive layer is essentially nil.

The powders for the adhesive layer powders may be any known material, such as CARBOPOL®. For the nonadhesive layer, calcium caseinate, sodium caseinate, and fresh curd casein are all effective. Flavor may be added with a small amount of sucralose or glycyrrhizin or powdered mint oil or thaumatin. A glidant, such as amorphous silica helps the flow. A binder such as gum arabic or hydroxypropylcellulose may be added to make the layer stronger.

With 12-14 mm flat or nearly flat punches or a flat lower punch and a slightly concave upper punch, press 80-120 mg pure gelatin granules for the adhesive layer with 0.5 to 1.5 tons pressure. Then add 40-80 mg pure calcium caseinate and press with 0.5 to 1.0 tons more pressure than the first pressing.

Patches made by this method were tested for mouth feel by four testers who judged the slipperiness and the duration of the casein layer to be excellent.

Several variations were effective as well. In variation 1, the nonadhesive layer contained up to 1% sucralose. In variation 2, the nonadhesive layer contained up to 4% hydroxypropylcellulose. In variation 3, the nonadhesive layer contained up to 4% gum Arabic. In variation 4, the nonadhesive layer contained up to 2% amorphous silica. In variation 5, the nonadhesive layer is constructed such that after the patches are made, a mist of water is applied to the casein side and dry, restraining the patches to prevent curling. This process causes the casein layer to become very strong.

### Example 2

### Patches Made by Depositing an Adhesive Layer and Dusting on a Casein Layer

With a depositor, such as an NID junior, deposit hot blobs of gelatin and water onto sheets of plastic. Before the blobs cool and gel, drop powdered calcium caseinate from a duster onto each blob to which the powder will adhere due to wetness and gelation. Allow to finish cooling and gelling (3-30 minutes), and then remove casein that is not adhered to blobs by blowing with air or brushing and tilting the sheet for a gravity assist. Then drop a fine mist of water with 0.2% glycerin for plasticizer sufficient to wet all the casein particles. Then dry the sheet to remove water simultaneously from both the gelatin layer and the casein layer so that the blobs form patches with tapered edges and a thin layer of strong, flexible casein film. Then heat seal a layer of plastic or foil onto the sheet and around each patch and die cut the resulting sandwiches.

Patches made by this method were tested for mouth feel by two testers who judged the slipperiness and the duration of the casein layer to be excellent. The patch thickness was thin enough to be very flexible and unobtrusive.

### Example 3

### Patches Made by Molding Both Adhesive and Casein Layers

Set up a machine with a die plate having 12-13 mm round holes and upper and lower flat faced punches that move up and down in the holes. Set lower punches to create recesses in the die plate of a preferred depth and scrape into each recess a paste (crumble) of casein and 25-30% water. Insert upper punches and press the casein crumble flat. Remove upper punches. Adjust height of lower punches to create recesses in the die plate of a preferred depth and scrape dry powdered gelatin into the recesses. Lay a sheet of plastic over the recesses and press from above with a flat upper plate and tightly press the die plate against the flat upper plate with the plastic in between. Then raise the lower punches and press the casein and gelatin layers against the sheet of plastic backed by the flat plate. The water from the casein will cause both the casein and the gelatin to sinter. Heat or apply vacuum or both until substantially all water is evaporated. Turn over to keep gravity in the best orientation. Lift the die plate while holding down the punches to keep the patches pressed against the plastic. Remove the die plate and then the lower punches to have bilayer patches adhered via gelatin to the sheet of plastic. Then heat seal a layer of plastic or foil onto the sheet and around each patch and die cut the resulting sandwiches.

Patches made by this method provided the strongest and longest lasting casein layer when tested in the mouth.

### Example 4

### Patches Made by Tablet Pressing Adhesive Layer and Printing Casein Layer

With 12-14 mm flat or nearly flat faced punches, press 80-120 mg of adhesive granules for the adhesive layer with 1 to 3 tons pressure. Hold the patches in a frame consisting of either a flat plate no thicker than the patches with a hole for each patch into which the patches are held by gravity or a hexagonal frame into which many patches fit closest packed arrangement. Place the frame under a screen printer or ink-jet printer or rubber transfer printer and print the exposed side of each patch with calcium caseinate mixed with 11-24% casein, 75-90% water, and 0-1% glycerin as plasticizer. In one pressing, 20% casein and 80% water were used. Then dry. A drug spot may be printed on the other side of the patch before or after the casein layer is applied.

Patches made by this method were tested for mouth feel by testers who judged the slipperiness and the duration of the casein layer to be excellent. The casein layer is very strong.

### Example 5

### Patches Made by Die Cutting Adhesive Layer and Printed Casein Layer

Form a sheet of mucoadhesive material as is well known by those skilled in the art. Paint one side with casein paint by placing the sheet face down on a puddle of casein paint or passing it over a roller of casein paint or by placing it under a sprayer or screen printer or rubber transfer roller and printing with casein paint. The paint may be made from calcium caseinate mixed with 11-24% casein, 75-90% water, and 0-1% glycerin as plasticizer, preferably 20% casein and 80% water. Then dry and die cut the sheet into patches. A drug spot may be printed on the other side of the patch before or after the casein layer is applied and before or after the sheet is die cut.

I claim:

1. A multilayer, mucoadhering patch, comprising
(a) a first, exposed layer of adhesive material including a polymer that is capable of adhering to a wet mucous surface in a human or other animal; and
(b) a second, exposed layer of nonadhesive material comprising casein.

2. The patch of claim 1, wherein the concentration of casein is at least 40% by weight of the nonadhesive layer.

3. The patch of claim 1, wherein the concentration of casein is at least 80% by weight of the nonadhesive layer.

4. The patch of claim 1, wherein the casein is selected from the group consisting of acid casein, rennet casein, fresh curd casein, micellar casein, calcium caseinate and sodium caseinate.

5. The patch of claim 1, wherein the patch comprises two layers.

6. The patch of claim 1, wherein at least one of the layers is a pressed powder.

7. The patch of claim 6, wherein the first layer is a pressed powder and the second layer is painted onto one side of the first layer.

8. The patch of claim 1, wherein the first layer and the second layer are bonded together by holding in contact and curing at least one layer.

9. The patch of claim 8, wherein the patch is cut from sheets of the first and second layers that are bonded together by curing.

10. The patch of claim 1, wherein the first and second layers are cured by drying.

11. The patch of claim 1, wherein orthogonal dimensions of the patch are each greater than about 5 mm and less than about 18 mm.

12. The patch of claim 1, wherein orthogonal dimensions of the patch are each greater than about 5 mm and less than about 30 mm.

13. A kit, comprising a multilayer, mucoadhering patch, comprising
(i) (a) a first, exposed layer of adhesive material including a polymer that is capable of adhering to a wet mucous surface in a human or other animal; and (b) a second, exposed layer of nonadhesive material comprising casein; and
(ii) instructions for use.
